(19) United States
(12) Patent Application Publication
(10) Pub. No.: US 2010/0292586 A1
(43) Pub. Date: Nov. 18, 2010

(54) Wireless automatic ankle-brachial index (AABI) measurement system

(76) Inventors: Thom W. Rooke, Rochester, MN (US); David Liedl, Eyota, MN (US); Lieven Cauwenberghs, Mol (BE)

Correspondence Address: DOUGLAS L. TSCHIDA;Suite 202, 93 Little Canada Road West, St. Paul, MN, 55117-2369, US

(21) Appl. No.: 12/454,215

(22) Filed: May 13, 2009

(51) Int. Cl. A61B 5/02

(52) U.S. Cl. 600/492

(57) ABSTRACT

A central processor controlled system and test procedure for independently, contemporaneously and plethysmographically monitoring arterial blood pressure at a subject's arms and legs. Micro-controlled occluding and sensing cuffs containing sensors are inflated and deflated at each limb of a supine patient. Sensed AC and DC pressure data is wirelessly linked to the central processor where the DC sensor data is sampled to derive median filtered, fitted and derivative waveforms that are iteratively processed and scored to determine a table of sample indices indicative of lowest pressure point. Second scoring and fittings about the lowest pressure point at the DC sensor data and original occluding cuff pressure data identify each limb's systolic pressure. The derived systolic limb pressure values are then processed to determine right and left ABI values.

### BACKGROUND OF THE INVENTION

The present invention relates to devices for detecting peripheral vascular disease (PVD) and, in particular, to a modular, wireless, self-inflating/deflating cuff system measuring AC and DC coupled arterial pressure components for independently determining a systolic arterial pressure at each of a test subject's limbs (i.e. arms and legs/ankles) and from which an ankle-brachial index (ABI) value is determined that can be used in evaluating the subject's cardiovascular condition.

The subject assembly is used in the evaluation of peripheral vascular disease, in particular peripheral arterial diseases (PAD). PAD occurs when arterial vessels become occluded, partially occluded, or stenotic in the periphery. If left undiagnosed and/or untreated, the reduced flow condition(s) may lead to a higher risk of myocardial infarction, stroke, and cardiovascular mortality.

While there are many causes of PAD, the most common cause is atherosclerosis. Atherosclerosis occurs with the build-up of deposits of fatty substances, for example, cholesterol, cellular waste products, calcium and other substances at the inner lining of an artery. This buildup is called plaque and usually affects large and medium-sized arteries. Some hardening of arteries occurs naturally as people grow older.

Plaques can grow large enough to significantly reduce blood flow through an artery. The plaque can also become fragile and rupture. Plaques that rupture can cause blood clots to form that can further block blood flow and/or break off and travel to another part of the body. If either happens and blocks a blood vessel that feeds the heart, it causes a heart attack. If the clot blocks a blood vessel that feeds the brain, it causes a stroke. If the blood supply to the arms or legs is reduced, it can create difficulties in walking and in severe cases can eventually cause gangrene.

The Ankle-Brachial Index (ABI), also known as Ankle Pressure Index (API) or Ankle Arm Index (AAI), is widely used to assess peripheral arterial disease. The ABI-test provides a well-documented, indirect method of comparing the relation of blood pressure in the arm to the blood pressure in the ankle and from which an assessment of arterial blood flow can be determined. Simply stated, ABI is the ratio of systolic blood pressure at the limbs (i.e. ankles/legs versus brachial/arms) and the general equation for determining ABI is as follows:

$$A B I = \frac{Ankle Systolic Blood Pressure}{Brachial Systolic Blood {Pressure}^{*}}$$

$${}^{*} Highest systolic pressure found in left or right arm$$

ABI has been shown to have a direct suggestive correlation to PAD and also to have an inverse correlation to the risk of cardiovascular disease (CVD). As shown in the table below, the risk of cardiovascular disease is inversely proportional to the ABI score. That is, the lower the ABI score, the greater risk of cardiovascular disease. Generally accepted ranges of ABI ratios and symptomatic conditions are shown in the table below. It is to be appreciated, however, that ABI values and ranges are not absolute and each individual's symptomatic condition can vary. Moreover, ABI testing is merely one of several tests that might be conducted while evaluating a patient's cardiovascular condition. Other tests might comprise stress testing, Doppler testing, ultrasound testing, among still others.

| ABI Ratio | Consideration |
|---|---|
| 0.96 or above | Generally Normal |
| 0.81-0.95 | Indicates mild, possibly asymptomatic disease |
| 0.51-0.81 | Indicates moderate disease |
| 0.31-0.50 | Usually indicates severe, multilevel occlusive |
| | disease |
| 0.30 or below | Severe disease. Usually indicates ischemic rest |
| | pain or tissue loss |

Source: The Cleveland Clinic, Department of Cardiovascular Medicine, Cleveland, Ohio and Techniques in Noninvasive Vascular Diagnosis: Protocol and Procedures Guideline Manual. R. J. Daigle BA, RVT. Academy Medical Systems 1999. p. 134

ABI ratios are calculated by monitoring the arterial pressure of each of the right and left ankles and dividing the detected pressure by the highest brachial pressure found between either the left or right arm. Consequently for each exam, a Right ABI index value (i.e. right ankle pressure/highest arm pressure) and a Left ABI index value (left ankle pressure/highest arm pressure) is determined. The “highest” arm pressure is used in both calculations and all calculation are typically presented in mmHg (i.e millimeters of Mercury).

A variety of techniques and devices have been developed to measure ABI. Representative measurement methodologies include cuff/stethoscope auscultatory methods, oscillometric methods, photoplethsmography, and Doppler ultrasound methods, among others.

The subject invention provides an automatic system and four pneumatic cuff monitoring assemblies for conveniently and contemporaneously measuring systolic arterial limb pressures. The pressures are primarily used to determine a patient's ABI value. The assemblies can also be used for other purposes. The measured ABI values and arterial pressures are considered and reviewed by qualified diagnosticians for accuracy and utility relative to the subject's cardiovascular health condition. For certain patients, especially those with weak limb blood flow, meaningful data may be difficult to obtain.

The subject invention provides a convenient system and assembly for obtaining a patient's ABI values. In a matter of minutes, the system generally performs a multi-limb plethysmographic measurement and diagnostic test. Four cuff assemblies are separately mounted to a subject's arms and ankles. A central processor (e.g. computer or any of a suitably programmed variety of portable or stationary signal/data processing devices) independently communicates over a wireless link with each cuff assembly. Self-directed inflation and deflation control signals direct pump and cuff operation. Heart pulse/beat data is sensed and stored in a local memory over a succession of stepped deflation pressure levels and communicated to the central processor.

The central processor evaluates the time/pressure data and during which the data is sampled and several indexed or addressable tables of sample values defining mean amplitude and derivative waveforms are derived. A variety of smoothing, fitting and scoring operations are performed on the sampled data to detect and remove artifacts (e.g. from the test procedure, electrical noise, subject motion) prior to determining relevant systolic pressure values for each monitored limb. The derived systolic limb pressure values are then used to determine right and left ABI values for a test subject.

### SUMMARY OF THE INVENTION

It is a primary object of the present invention to provide a low-cost, modular ABI measurement system for automatically and independently measuring arterial pressures at a patient's limbs and automatically determining relevant ABI index values.

It is a further object of the invention to provide a wireless system for controlling the inflation and deflation of multiple monitors mounted to each of a subject's four limbs and determining relevant systolic pressures.

It is a further object of the invention to provide each monitor with micro-controlled sensing and occluding cuffs, a compressor, inflation/deflation valves, pressure sensors, data storage and communications capability for occluding and sensing relevant pressures and blood flow.

It is a further object of the invention to provide a central processor and associated memory for controlling system operation and computing right and left ABI values from AC and DC components of the sensed pressures.

The foregoing objects, advantages and distinctions of the invention, among others, are found in a central processor controlled system that directs wireless control and data transmissions to four, automatic monitors independently fitted to each of a subject's limbs. Each monitor assembly includes both occlusion and sensing cuffs that independently communicate with the central processor/controller. The automatic ankle brachial index system (AABI) generally performs a plethysmographic measurement of the systolic arterial pressure at each limb in a matter of minutes.

Control and data signal communications occur over appropriate communication link(s) (e.g. wireless) between the central processor and the occlusion/sensing cuffs of each monitor assembly. Each monitor assembly generally operates independent of the others. Self-directed inflation and deflation control signals direct pump and cuff operation. The sensing cuffs of the AABI system each inflate to a fixed, pre-set pressure (e.g. 30 mmHg) and remain inflated throughout each test exam. The occlusion cuffs each inflate to a pressure at which the sensing cuff stops sensing a pulse volume measurement (PVR) signal or to a maximum pressure 20 mmHg greater than the sensed PVR pressure or to a preset maximum pressure (e.g. 250 mmHg), whichever occurs first.

The AABI then pauses to allow stabilization of pressure values. The occluding cuffs are deflated at an approximate rate of 1 mmHg/second. During deflation of the occlusion cuff, AC (i.e. rapid pulsation) and DC (i.e. static or slow pulsation) components of the sensed blood flow signals are monitored until a return a normal pulsed blood flow is detected.

Following the complete deflation of each occlusion cuff, preprogrammed software algorithms in the central processor/controller of the AABI system analyzes the sensed AC and DC components of the pressure/time data to determine a systolic arterial pressure for each limb. Generally, the software looks for the point where the static pressure begins to increase. Upon determining a relevant pressure slope transition from a number of indexed or addressed sample data tables, a pressure measured at the occlusion cuff and correlated to a low point index at the sampled data waveforms is reported in mmHg as the systolic arterial pressure to be used in the calculation of the relevant right and left ABI values. The measured ABI values and related oscillometric waveforms are displayed along with other relevant test data at an associated monitor or test report.

Still other objects, advantages and distinctions of the invention will become more apparent from the following description with respect to the appended drawings. Considered alternative assemblies, methodologies, improvements and/or modifications are described as appropriate. The description should not be literally construed in limitation of the invention. Rather, the scope of the invention should be broadly interpreted within the scope of the further appended claims.

### BRIEF DESCRIPTION OF THE DRAWINGS

Similar reference numerals and characters at the drawings refer to like structure at the various drawings and which are as follows:

FIG. 1 shows a system diagram wherein four limb monitors (only one of which is shown) communicate with a controller/processor over a communications link (e.g. wireless) and wherein each monitor is comprised of a set of portable sensing and occluding blood pressure cuffs and associated circuitry.

FIG. 2 shows a generalized block diagram to the system operation.

FIG. 3 shows a block diagram to the sensing cuff inflation cycle.

FIG. 4 shows a block diagram to the occluding cuff inflation cycle.

FIG. 5 shows a block diagram to the occluding cuff deflation cycle.

FIG. 6 shows a pressure versus time waveform to the inflation, deflation and sensed pressures at the sensing cuff.

FIG. 7 shows a pressure versus time waveform to the inflation, deflation and sensed pressures at the occluding cuff.

FIG. 8 shows a plethsymographic waveform for limb data received from a test on a subject with no peripheral arterial disease (PAD), no artifacts and a mean arterial systolic pressure of 119.3 mmHg as determined by the software preprogrammed into the central processor.

FIG. 9 shows a plethsymographic waveform for limb data received from a test on a subject with mild peripheral arterial disease (PAD), a very noisy center line wherein the artifacts may be due to calcification in the vessel, and a mean arterial systolic pressure of 98.3 mmHg.

FIG. 10 shows a plethsymographic waveform for limb data received from a test on a subject with severe peripheral arterial disease (PAD), some motion artifacts at the center line, and a mean arterial systolic pressure of 54.3 mmHg.

FIGS. 11 and 12 show block diagram flow charts to the process and sequence of steps performed by the central processor and system software to search for the lowest point where the static pressure begins to increase and which defines the systolic arterial pressure used in the ABI calculations.

FIG. 13 shows a sampled median waveform of the DC component of the original pressure data in dashed line sensed by the sensing cuff 14 and a fitted or smoothed sampled waveform of the original data in solid line.

FIG. 14 shows a derivative of the fitted waveform of FIG. 13 to the DC component of the pressure data sensed by the sensing cuff 14.

FIG. 15 shows a subset of the original DC pressure waveform for the occluding cuff taken around a calculated lowest point index and from which the limb's systolic pressure is determined.

FIG. 16 shows a typical representation of a multi-window test report as displayed on a monitor screen and depicting the derived right and left ABI index values and related blow flow data sensed for the examined subject.

FIG. 17 shows a schematic diagram of the microprocessor based control circuitry of each cuff assembly.

FIG. 18 shows a schematic diagram to the band pass filters of each cuff assembly.

FIG. 19 shows a schematic diagram to the pump driver circuitry used to inflate and deflate the sensing and occlusion cuffs 14 and 16.

FIG. 20 shows a block diagram to an occluding cuff inflation cycle when the occluding cuff is used to sense pulse volume (PVR) data.

### DESCRIPTION OF THE PREFERRED EMBODIMENT

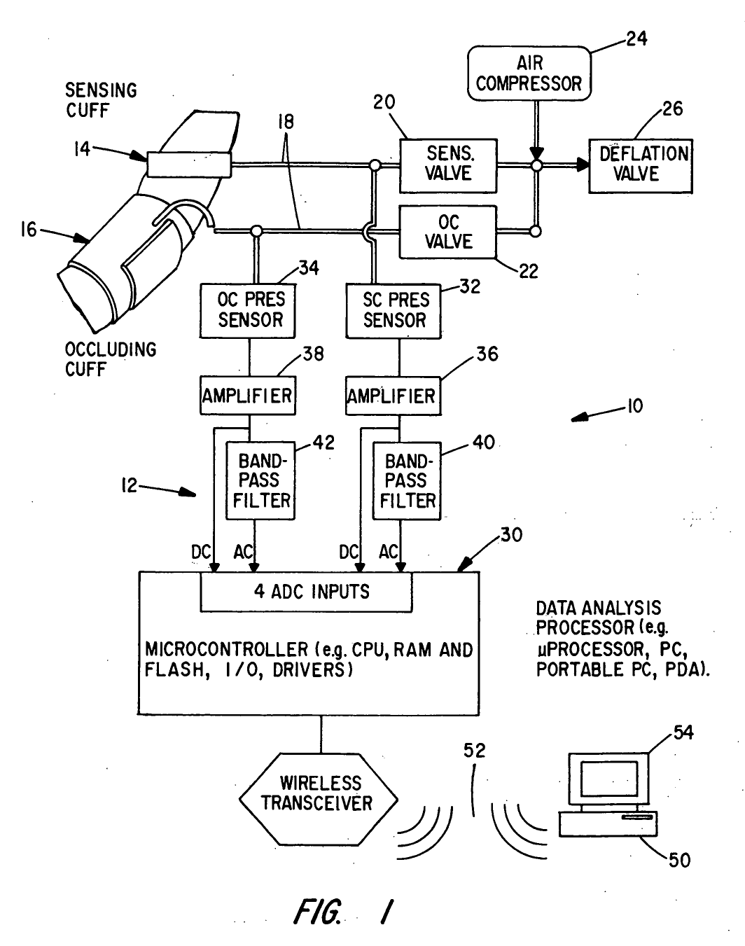

With attention to FIG. 1, a generalized overview is shown to the automatic ankle brachial index measurement system (AABI) 10 of the invention. The system 10 is depicted in relation to a single cuff monitor assembly 12 which is shown in an exemplary mounting to an arm. It is to be appreciated however the system 10 when performing an ABI diagnosis on a typical subject uses four identical monitors 12. One monitor assembly 12 is coupled to the extremities of each of a subject's limbs. That is, a monitor 12 is coupled to each arm and to each leg.

Each monitor assembly 12 comprises a pair of portable, inflatable sensing and occlusion cuffs 14 and 16 which are respectively constructed to perform sensing and occlusion functions. The cuffs 14 and 16 can be constructed to any desired shape and size to accommodate the limb and task to be performed. The cuffs 14 and 16 are cloth covered and include an internal bladder (not shown). The cuffs are inflated and deflated via associated supply conduits 18, inflation valves 20 and 22, air compressor 24 and deflation valve 26. The cuffs 14 and 16 include appropriate fasteners (e.g. overlapping hook and loop fasteners) to securely attach to a limb (e.g. upper arm, leg or ankle) or appendage (e.g. wrist, finger or toe). Presently, the sensing cuffs 14 are constructed to be slightly smaller than the occluding cuffs 16 to facilitate attachment to the regions of sensing at the limb extremities.

Each set of portable sensing and occluding cuff assemblies 14 and 16 are coupled to monitor control circuitry 30 (i.e. monitor controller) via intermediate sensors 32, 34, amplifiers 36, 38 and filters 40, 42 (e.g. band pass). The sensors 32, 34 are incorporated into the cuffs 14 and 16. Upon inflation of the associated cuffs 14 and 16, the sensors 32, 34, detect and produce electrical signals containing direct (DC) and alternating/pulsed (AC) signal components. The detected AC and DC signals are amplified and the components are coupled to appropriate input ports 44 of the controller 30.

The sensors 32, 34 can be constructed from any of a variety of devices that can sense changes in a physical condition and produce a related electrical signal. For example, piezoelectric elements, strain gauge or optical assemblies are able monitor and convert physical movements at the subject to electrical signals. Preferably any selected pressure measuring device is adaptable to a cuff mounting.

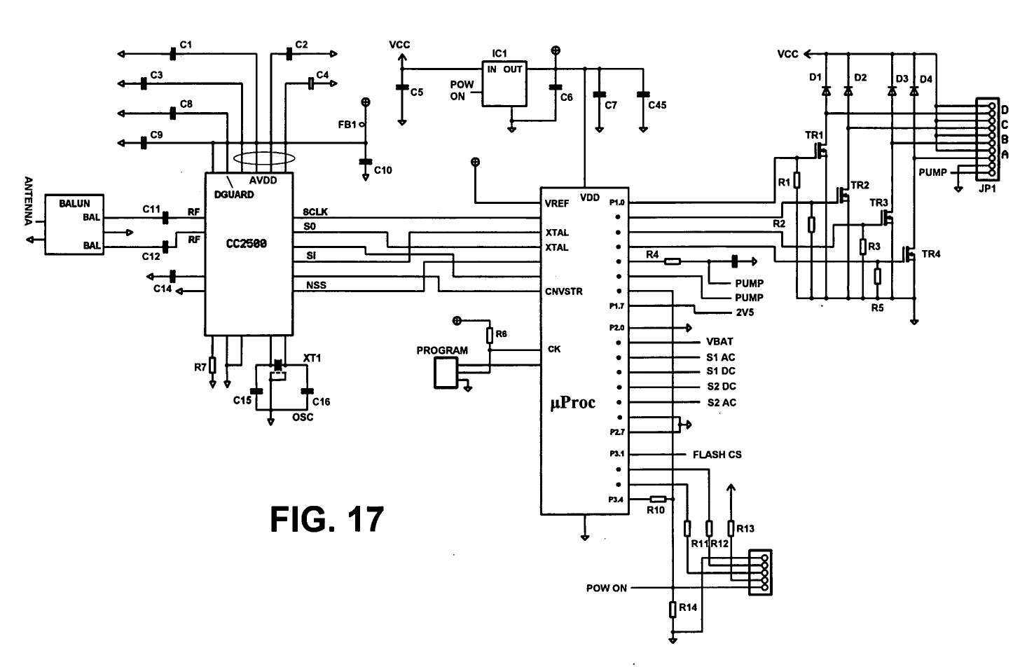

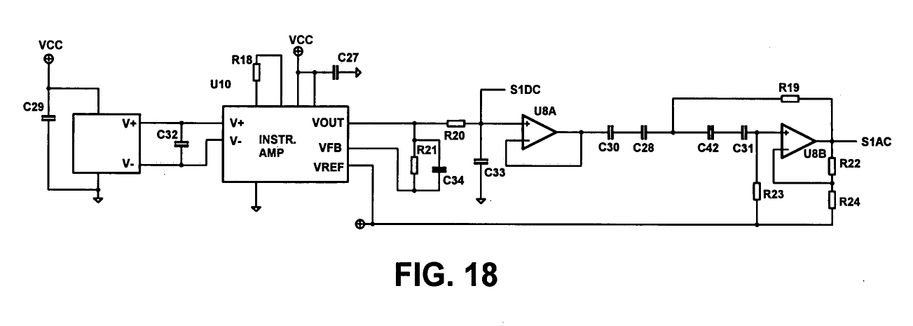

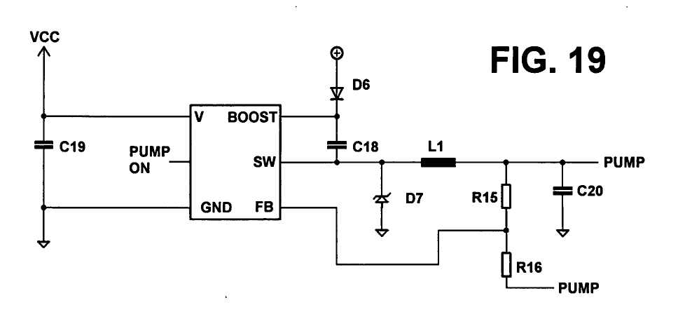

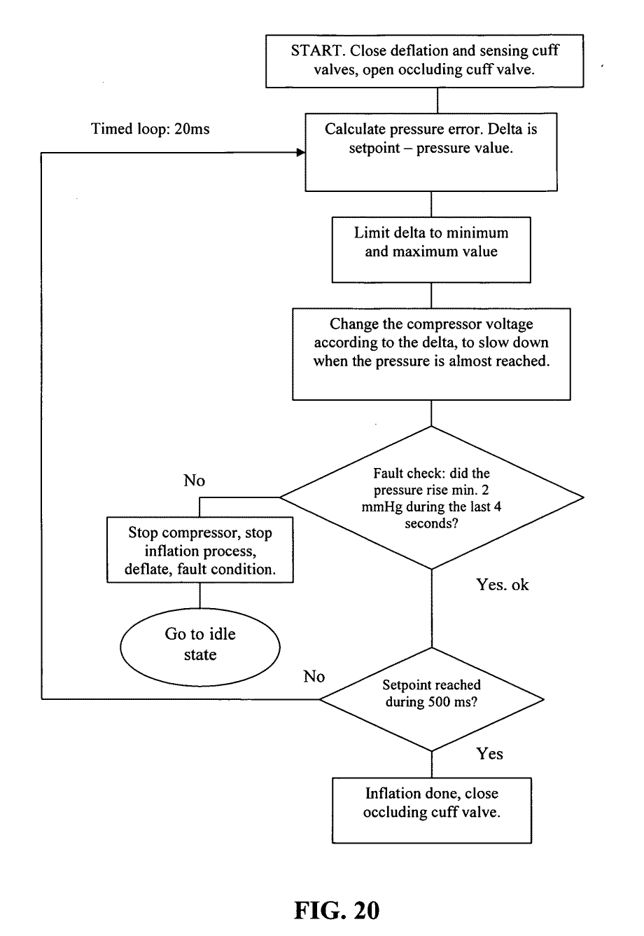

The monitor controller circuitry 30 includes a processor unit (e.g. microprocessor/CPU), associated storage memory (e.g. RAM, ROM, flash) of suitable type and configuration, drivers and input/output (I/O) circuitry to communicate with a primary or central processor 50. The controller 30 responds to a preprogrammed or programmable instruction set to automatically control the operation of each monitor assembly 12. A detailed schematic to the controller 30 is shown at FIG. 17. Detailed schematics to the band pass filers 40 and 42 are shown at FIG. 18 and a detailed schematic to the pump driver circuitry is shown at FIG. 19.

The monitor controller 30 also includes I/O circuitry that communicates over a suitable communications link 52 (e.g. wired network, phone system, wireless system, WIFI etc.) with the primary processor 50. The processor 50 is coupled to a display monitor 54 or other suitable device that displays and communicates relevant test information to an operating technician (e.g. CRT, LCD/TFT screen, or printer). Depending upon the system 10, the processor 50 can comprise a portable or stationary PC, a mid-sized or mainframe central computer, PDA, special purpose handheld device or any other device containing an appropriately programmed processor with supporting storage memory, communications capabilities and sundry other devices. The processor 50 is operative to perform the necessary interpolation of the test data and display the operation of each monitor 12 and the results of each diagnostic test at the monitor 54.

The processor 50 presently comprises a portable, laptop computer with suitable processing power and communications capabilities. Presently a wireless network connection 52 is established with each of the cuff monitor assemblies 12. The processor 50 might also be coupled to the monitors 12 via other wired or wireless network or internet connections and/or to other larger systems where the test data is stored in a database.

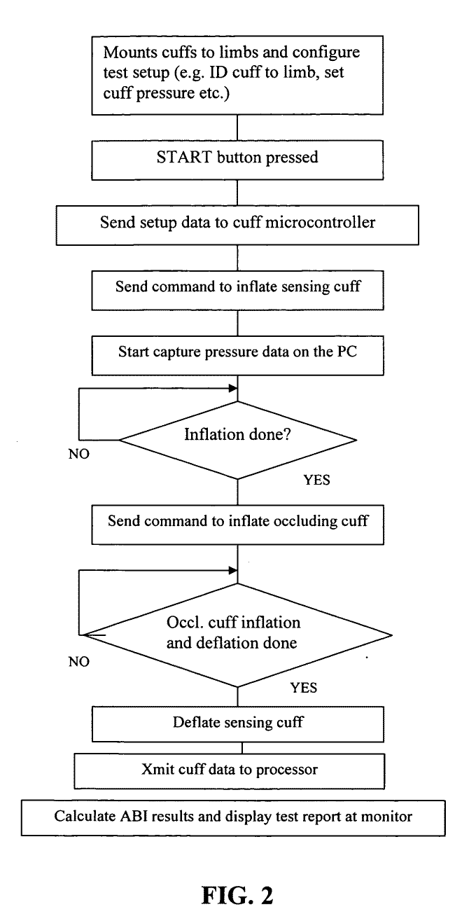

With attention next directed to FIGS. 2 through 6, the AABI System 10 provides an automatic, plethysmographic method to measure appropriate systolic limb pressures for the purpose of obtaining a number of data points from which the subject's relevant ABI index values are derived. The system 10 was developed for ease of handling and operation by diagnostic personnel (e.g. nurses, medical technicians etc.), yet provides high sensitivity and accuracy. Each cuff monitor assembly 12 provides for an indirect (noninvasive) measurement of arterial blood pressure. Each cuff monitor assembly 12 automatically inflates and deflates its associated cuffs 14 and 16 and determines the systolic blood pressure for each limb from signals obtained via a suitable pressure sensor or transducer fitted to each of the sensor and occlusion cuffs 14 and 16.

The cuffs 14 and 16 are of generally conventional construction and each is operative to expand and collapse with the movement of supplied and vented air. Micro-programmed operating and signal processing software instructions described below relative to the flow charts of FIGS. 2 through 5, 9 and 10, control operation of the system 10. It is also to be noted that a common battery charger can be provided with the system whereby all four monitor assemblies 12 can be simultaneously charged between tests.

Each test is performed by first placing the patient in a supine or horizontal position. The supine position places the arms, ankles and cuff monitor assemblies 12 at the same horizontal level as the heart. This position also tends to reduce motion artifacts and isolate systolic pressure variations to accurately reflect the subject's vascular condition. The monitor assemblies 12 are next mounted to the subject's limbs.

The larger occluding cuffs 16 are mounted to a patient or subject's right and left upper arms and right and left calves or legs in the region of the ankle. The smaller sensing cuffs 14 are mounted to the wrists or fingers and ankles or toes. Each monitor assembly 12 is assigned a unique digital ID code and once mounted; the processor 50 as it receives data identifies each cuff 14 and 16 to the respective limb to which it is attached.

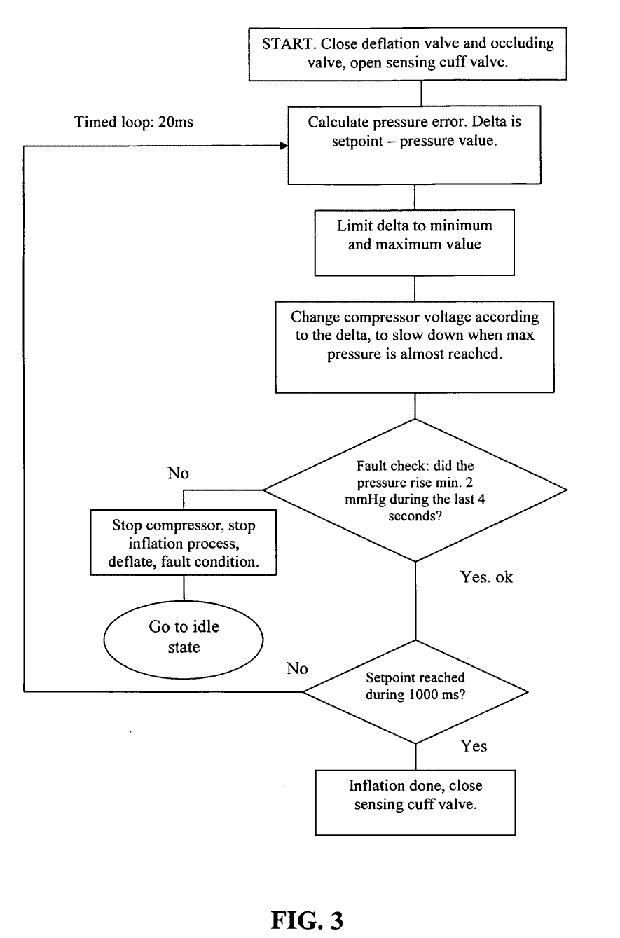

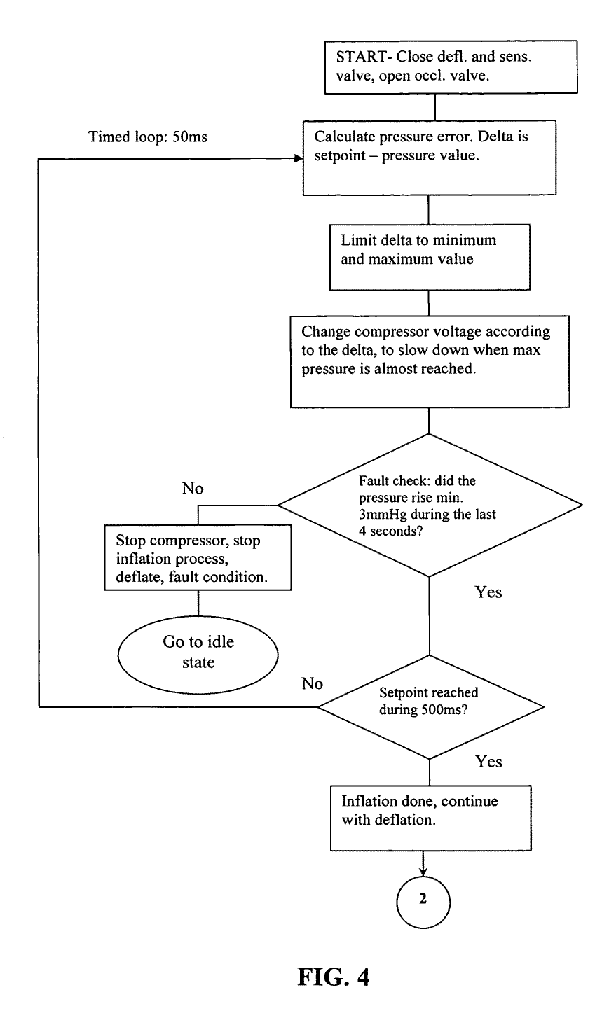

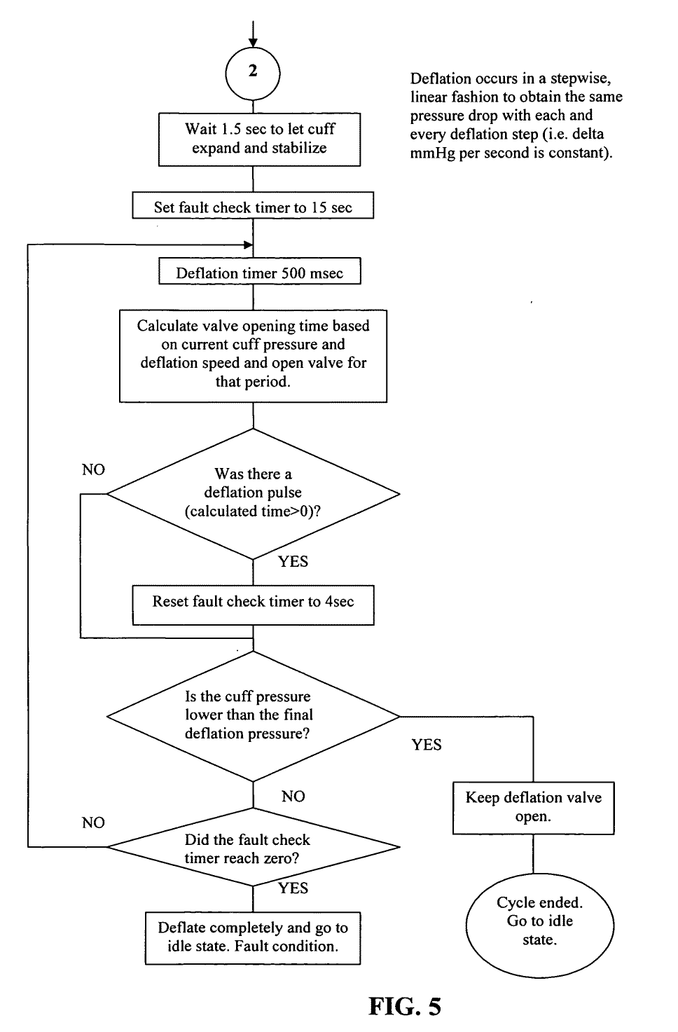

FIG. 2 shows a flow chart to the general sequence of steps performed with each test at each monitor 12. A more detailed flow chart to the inflation of the sensing cuff 14 is shown at FIG. 3. FIG. 4 shows a detailed flow chart to the inflation of the occluding cuff 16; and FIG. 5 shows a detailed flow chart to the deflation sequence of the occluding cuff 16 during the sensing phase of a test cycle and deflation of both cuffs 14 and 16 upon completion of the test.

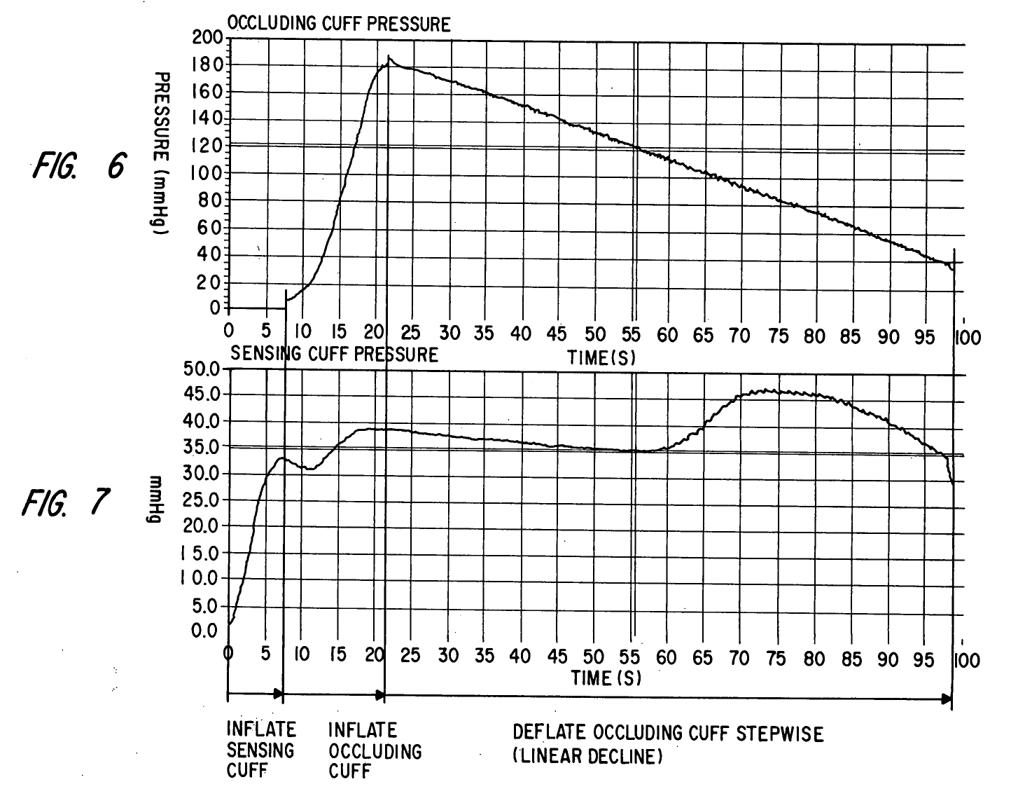

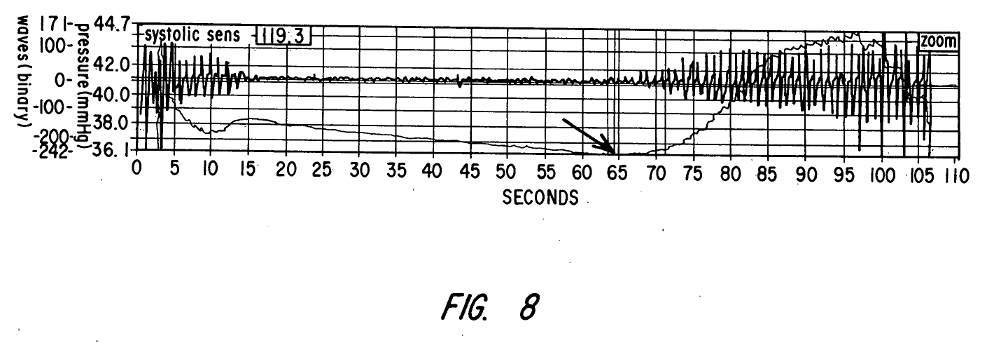

FIGS. 6 and 7 show exemplary pressure versus time waveforms for pressures sensed during a test of a limb at the occluding and sensing cuffs 16 and 14. FIG. 8, in turn, depicts a typical composite plethsymographic waveform for the limb under test at FIGS. 6 and 7. The composite waveform of FIG. 8 is generated by the processor 50 upon processing the sensed DC and AC components of the pressure data signals using signal processing software preprogrammed into the processor 50. Similar waveforms are obtained for each limb tested and from which “mean arterial pressures” are determined for each limb to compute a test subject's relevant ABI index values.

With attention to FIG. 2, the sensing cuff 14 is first inflated to a pressure sufficient to assure intimate contact with the associated pressure transducer (e.g. 30 mmHg). The occluding cuff 16 is then inflated to a pressure sufficient to occlude the artery and pulsed flow. The occluding cuff 16 is then deflated in an incremental step-wise fashion until normal pulsed flow returns to the limb. A generally linear deflation sequence with equal pressure drops at each step is presently performed. During deflation the AC and DC pressure signal components are sensed by the cuffs 14 and 16 and communicated to the controller 30.

Pertinent micro-programmed instructions that control cuff inflation/deflation and data collection are stored in included RAM, ROM and/or flash memory or other associated memory coupled to the microcontroller 30 at each monitor assembly 12. All communication control and data signals, exclusive of data collection, are transmitted over the wireless link 52 between the processor 50 and each monitor assembly 12. Each transmission includes a preamble code, cuff identification code, relevant data and error checking data. The particular communication protocol can be varied as desired. The test results for each limb are organized in a database or tabular form and can be presented and documented in any desired printed or monitor displayed report(s).

Each microcontroller 30 controls the inflation and deflation process of its associated cuffs 14 and 16. Raw blood pressure data is sent to the primary processor 50 over the wireless link 52 from each set of cuffs 14 and 16. System calculations (e.g., blood pressure, pulse, etc.) are executed by the processor 50 using pre-installed software written in the LabVIEW language of National Instruments®. The micro-programmed instructions and signal processing software can alternatively be written in any other suitable language. A detailed description is provided below to the operation of the system 10 and processor 50 in relation to test data obtained from one limb. Similar sequences are performed for each of the subject's four limbs.

### Inflation Process

With attention to FIGS. 1 through 3 and upon attaching the monitors 12 to the test subject's limbs and placing the subject in a supine condition, a test “start” switch is initiated and the test setup data and instructions are sent to each microcontroller 30, although the following description is directed to a single monitor 12. The deflation and occluding valves 22 and 26 are closed, the sensing cuff valve 20 is opened and the air compressor 24 is engaged to inflate the cuff 12 to a set point pressure of approximately 30-40 mmHg. The microcontroller 30 is also enabled to transmit sensed pressure data to the processor 50. The sensing cuff 14 is inflated at a steady rate until just before the set point pressure. The compressor 24 is then slowed until the set point pressure is reached, when the compressor 24 is idled and the sensing cuff valve 20 is closed.

With the closing of the sensing cuff valve 20, the occluding cuff valve 22 is opened and air is admitted into the occluding cuff 16. A maximum inflation or set point pressure is automatically established at the initiation of each test by the system software and is typically set at approximately 150% of the maximum pressure at which peak arterial pressure is sensed by the cuff 14. A default, maximum inflation pressure (e.g. 250 mmHg) limited by the capacity of the compressor 24 or related equipment standards is also programmed into the monitor assembly 12. During each test, each occluding cuff 16 is inflated to occlude the brachial artery in the arms and the femoral artery in the legs.

Assuming a nominal maximum pressure range of 180-250 mmHg, the pressure at the cuff 16 is monitored during inflation relative to the above range to regulate and slow the compressor 24 as the maximum set point pressure is approached. The sensed pulsed flow AC pressure signal at the sensing cuff 12 is also monitored to determine the occlusion of flow in the limb. With a confirmation of occlusion at a pressure in the preset range, the microcontroller 30 stops the compressor 24. After a few seconds to permit the pressures to stabilize, the microcontroller 30 opens the deflation valve 26 and begins to deflate the occluding cuff 16 in a stepwise manner.

Because the occluding cuff 16 contains pressure sensing circuitry similar to the cuff 14, the cuff 16 can be used during certain tests to sense AC limb pressures. FIG. 18 depicts a PVR inflation sequence similar to FIG. 4 in the event the microcontroller 30 is configured to use the occluding cuff to sense AC pressure activity

### Deflation Process

FIG. 5 depicts a flow chart to the steps performed by the microcontroller 30 during the deflation phase. FIGS. 6 and 7 correspondingly depict the relative inflation and deflation pressures sensed at the cuffs 16 and 14 over a test sequence. During the deflation phase, the occluding cuff 16 is particularly deflated in a stepwise fashion over a series of equal pressure drops. Pulse width modulated signals are continuously calculated and applied to control the open time of the deflation valve 26 to achieve this end.

As the cuff 16 deflates, normal pulsed blood flow progressively returns to the limb. During each deflation step pulsed blow flow signals are progressively detected as the cuff pressure is released. The return of pulsed flow is better shown in the test data of FIG. 8. With pulsed flow returning and the pressure at the cuff 16 falling below a preset final deflation pressure, the deflation valve 26 is held open to release any remaining air from the cuff 16. The compressor 24 is idled and the test is completed.

As air is released from the occluding cuff 16, the pressure transducer at the occluding cuff 16 monitors the static cuff pressure. The pressure transducer at the sensing cuff 14 contemporaneously senses the gradual return of pulsed blood flow to the limb as the arteries re-expand. The static DC pressure at the cuff 16 and the pulsed AC pressure at the cuff 14 are particularly monitored and contemporaneously coupled to the processor 50. The processor 50 processes the data to determine the point in time where the static pressure at the sensing cuff 14 reverts from a declining pressure slope to an inclining slope and nominal pulsed flow returns. The processor 50 filters out extraneous pressure variations and slope changes to identify the primary or dominant slope change and related pressure at the waveform of FIG. 6 as the relevant systolic pressure. This pressure is used in the determination of the subject's ABI index values. For the test of FIGS. 6 and 7, the processor determined the systolic pressure occurred approximately 55 seconds into the test. For the test of FIG. 8, the systolic the pressure of 119.3 mmHg and slope reversion was determined to occur approximately 65 seconds into the test. The systolic pressure for both tests is determined from waveform data similar to that of FIG. 6.

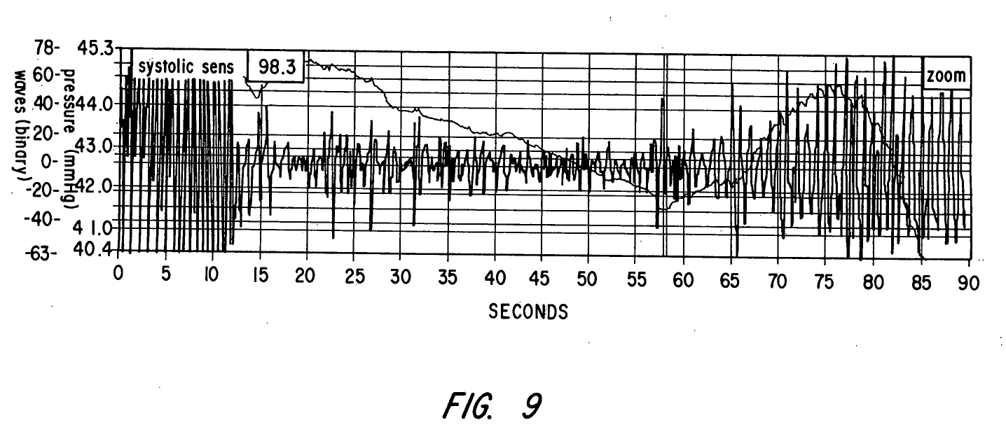

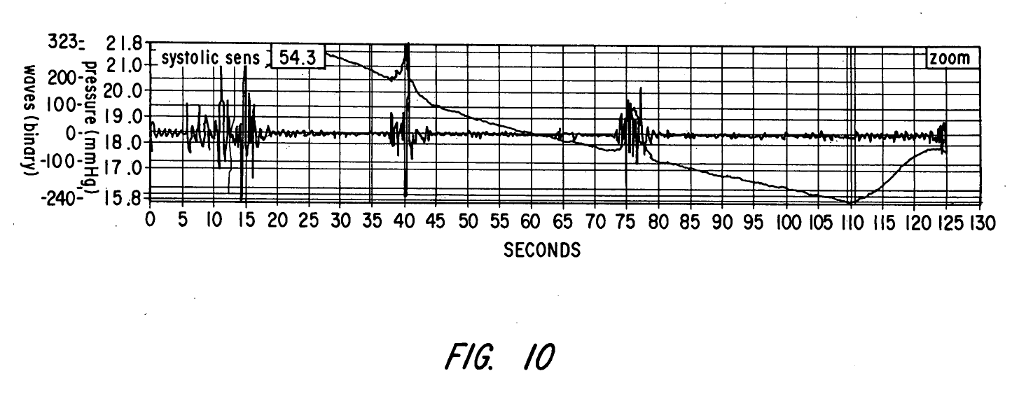

The test waveforms displayed at FIGS. 6-8 are representative of persons having generally healthy vascular systems and who did not move during their tests. The waveforms are therefore relatively free of artifacts. For a variety of reasons, such as less healthy subjects with occluded arteries or subjects that move or tense their muscles during a test, numerous pressure artifacts can be detected that produce several negative to positive and positive to negative slope changes in the sensed DC signals. FIGS. 9 and 10 depict test pressure waveforms for individuals with possible vascular blockage and/or who produced motion artifacts. The signal processing software described below is adapted to inspect each of these conditions and isolate the true negative to positive slope change and the related systolic pressure measured at that point by the occluding cuff 16.

### Data Processing

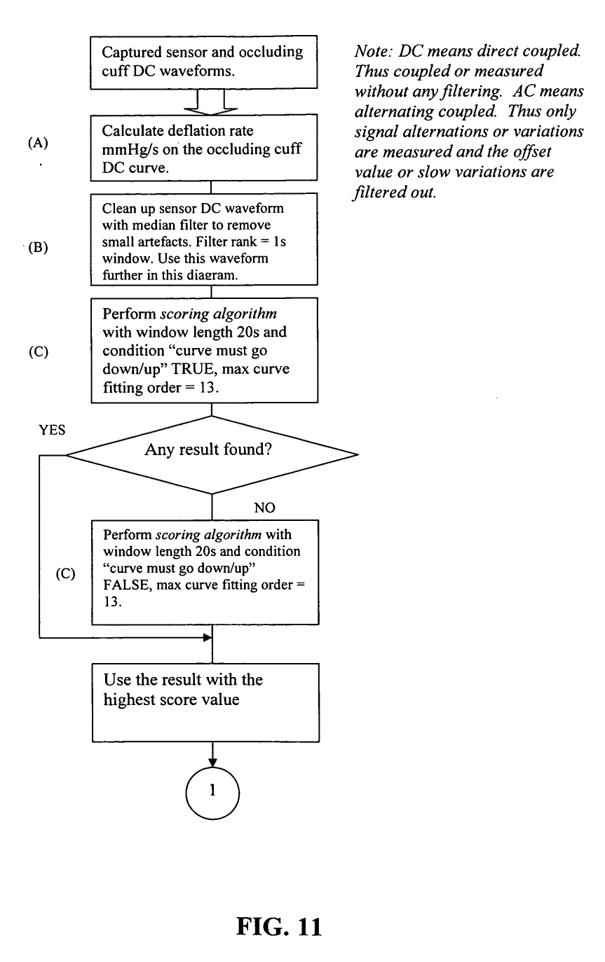

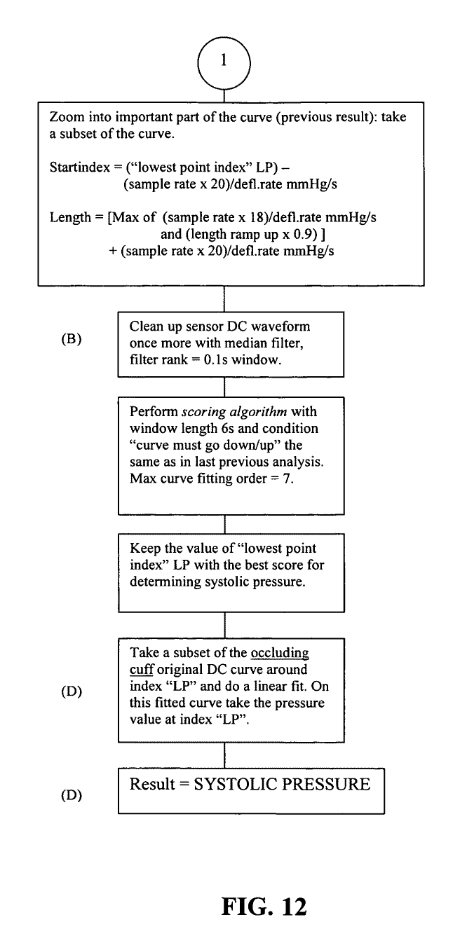

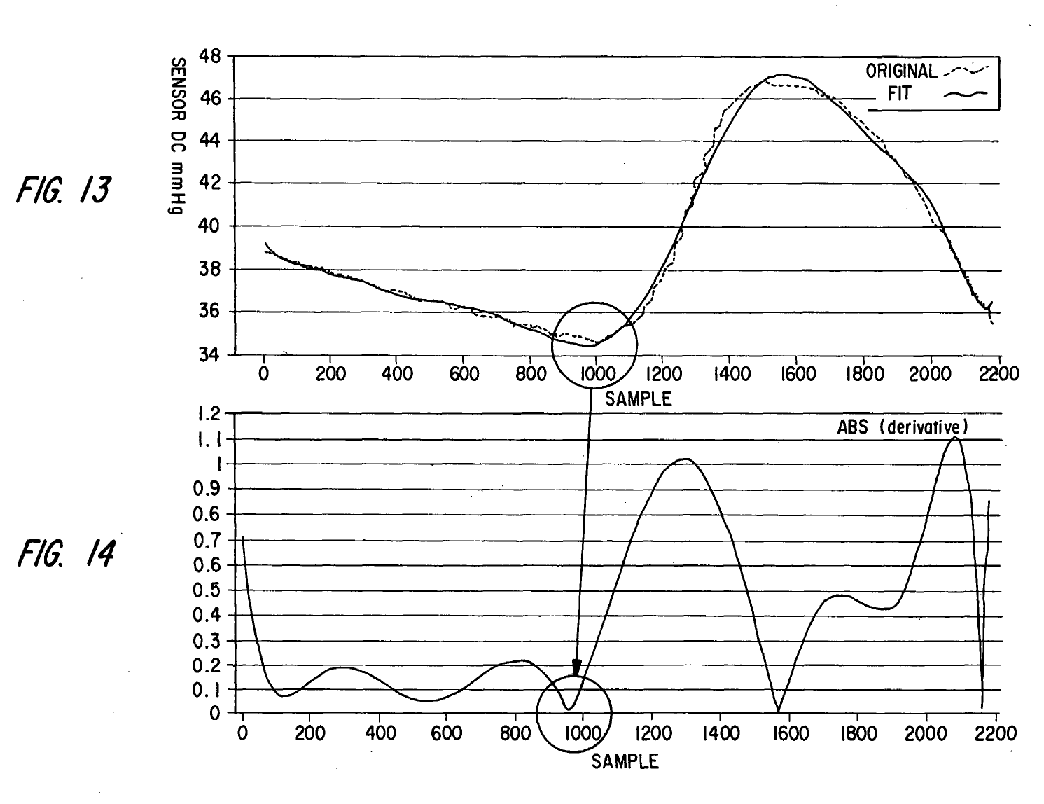

FIGS. 11 and 12 depict a flow chart to the steps performed by the processor 50 during the processing of the DC signals captured at the occluding and sensor cuffs 16 and 14. With the collection and transmission of each limb's AC and DC test pressure data, the primary processor 50 performs a series of iterative sequences of filtering, curve fitting, derivative computations and scorings to identify and quantify the pertinent systolic blood pressure at each limb tested. The four systolic pressures are then applied to determine a related ABI value for the test subject. FIG. 13 depicts filtered data compiled upon repetitively sampling the DC or static pressure data of FIG. 6 sensed by the sensor cuff 14 (shown in dashed line) and overlaid with polynomial fit data (shown in solid line). The original and fitted sample waveforms of FIG. 13 are analyzed to determine the “low point” (LP) (i.e. the index and/or address) of the sample where the waveform changes from a declining slope to an inclining slope.

FIG. 14 depicts a derivative waveform of the fitted DC sensor data of FIG. 13. The derivative waveform exhibits minimum or “zero” absolute values at each of the points where the fitted waveform of FIG. 13 transitions from negative to positive or positive to negative slopes. The processor 50 particularly examines the slope changes at these waveforms using a scoring algorithm described below to determine the lowest point LP in the associated sampled absolute value data.

The particular region of interest is the encircled portion of the waveforms in the region of the 1000^{th} sample although the complete waveform of FIG. 13 is inspected. With further examination of the circled region at the DC scored sensor data of FIG. 13, the processor 50 determines the index or address of the lowest or LP data point. The LP point of the DC sensor waveform 11 is lastly correlated back to the monitored occluding cuff pressure data at FIG. 7 to determine the physical pressure measured at the sample containing the indexed LP value. The measured pressure at the LP index is then used as the systolic pressure.

Initially and with attention re-directed to FIG. 7, the processor 50 subjects the DC occluding cuff data of FIG. 7 to a median filter having a rank of “1s” to remove minor artefacts and smooth the waveform. A sampling rate of 30 samples per sec in mmHg/sec is applied, although other rates can be used. The median filter is a standard function in the LabVIEW® application program sold by National Instruments and obtains the elements of Filtered X using the following equation.

yᵢ=Median(Jᵢ) for i=0, 1, 2, . . . , n-1,

where Y represents the output sequence Filtered X, n is the number of elements in the input sequence X, Jᵢ is a subset of the input sequence X centered about the i^{th} element of X, and the indexed elements outside the range of X equal zero. Jᵢ is given by the following equation.

Jᵢ={xᵢ₋ᵣ, xᵢ₋ᵣ₊₁ . . . , xᵢ₋₁, xᵢ, xᵢ₊₁ . . . , xᵢ₊ᵣ₋₁, xᵢ₊ᵣ}, where r is the filter rank.

The processor 50 then determines a “start index” and “last index” (i.e. sample number), where the “start index” coincides with the sample taken where the maximum pressure occurred when inflation was terminated and the “last index” coincides with the sample where the pressure was released from the occluding cuff 16. The processor 50 next determines the slope of the filtered DC pressure waveform for the occluding cuff 16. The slope or deflation rate mmHg/s is calculated as (mmHg at start index−mmHg at last index)/((last index−start index)/sample rate).

Using the “start” and “last” indexes determined above relative to the slope determination sampling of FIG. 7 (i.e. the maximum pressure and deflation pressure points of the occlusion cuff 16), the processor 50 next applies the DC sensor cuff 14 data of FIG. 6 to a similar median filtering step. A sampled set of data values, table and related waveform (shown in dashed line) is obtained and depicted at FIG. 11.

A curve fitting is next performed on the filtered or smoothed sample data for the sensor cuff DC pressure data waveform of FIG. 13 to obtain a relatively clean waveform from which the slope trends of the waveform can be more readily analyzed in further processing steps. The curve fitting is performed using the LabVIEW® “general polynomial fit” algorithm several times with incrementing values for the “order” input up to a “max fit order”. The data from the fitting sequences that provides the lowest mean square error (MSE) is stored and is displayed at FIG. 13 as the “best” fit waveform (shown in solid line). The “general polynomial fit” is a standard subroutine function found in the LabVIEW® software:
The following equation gives the general form of the polynomial fit.

$${f}_{i} = {\sum }_{j = 0}^{m} {a}_{j} \times {x}_{i}^{j}$$

where F represents the output sequence Best Polynomial Fit, X represents the input sequence X, a represents the Polynomial Fit Coefficients, and m is the polynomial order.
The VI calculates mean square error (mse) using the following equation.

$$m s e = \frac{1}{n} {\sum }_{i = 0}^{n - 1} {( {f}_{i} - {y}_{i} )}^{2}$$

where Y represents the input sequence Y and n is the number of data points.
The General Polynomial Fit VI is a special case of the General LS Linear Fit. The General Polynomial Fit VI uses the General LS Linear Fit VI as a subVI. The General Polynomial Fit VI builds the H matrix internally using input X for the General LS Linear Fit VI.
The following equations define the formula used to build H:

hᵢⱼ=fⱼ(xᵢ)=x^{j}ᵢ

i=0, 1, . . . , n-1

j=0, 1, . . . , m
For example,

$$H = [ \begin{matrix}1 & {x}_{0} & … {x}_{0}^{m} \\ 1 & {x}_{1} & … {x}_{1}^{m} \\ & ⋮ & \\ 1 & {x}_{n - 1} & … {x}_{n - 1}^{m}\end{matrix} ]$$

A derivative waveform shown at FIG. 14 of the fitted DC sensor data waveform of FIG. 13 is also determined using the derivative function at the same sampling rate

$$f ( t ) = \frac{d}{d t} F ( t )$$

where “dt” is 1/sample rate. The derivative waveform is sought as a check on where the slope trends of the fitted waveform of FIG. 13 change from declining to inclining and inclining to declining. The derivative waveform data is used during a scoring sequence applied to the waveforms to determine the low point (LP) index or sample value of the fitted waveform of FIG. 13.

A looped algorithm is next separately applied to the fitted DC sensor data waveform of FIG. 13 and to the derivative waveform of FIG. 14. Each waveform is iteratively examined in a looped fashion by selecting and examining “windows” of sequential groupings of the data values stored in the sampled data tables that define the waveforms of FIGS. 13 and 14. The number of loops or “loop index” is 2×(number of samples in curve)/(sample rate). The processor selects data windows of an appropriate width and each of which is presently set to be 20 data samples wide, although other widths can also be used. The start index of the window is equal to (loop index)×(sample rate)×0.5. The window width is defined as the (analysis length)×(sample rate)/(deflation rate). Other inputs to the scoring algorithm are the deflation speed and max fit order (e.g. 13).

The values of the sampled data stored at several addressable tables containing the index to each sample window are inspected to determine where the lowest data value in each window is located. Upon locating the lowest value, the processor 50 looks to a corresponding window of samples for the derivative waveform of FIG. 14 to confirm whether the located low value coincides with the approximate middle of the derivative window. The middle is defined as occurring in the range between 40% and 70% of the derivative window width. That is, the processor 50 seeks to correlate the sample low point of the fitted data point with a relevant sample zero point of the derivative waveform, since the zero or lowest points of the derivative waveform occur only where the slope transitions occur (i.e. down to up or up to down). If the answer is “yes”, the window is preliminarily accepted. If the answer is “no”, the processor 50 moves to the next window and loop iteration.

If the answer is yes, the sampled data of the derivative window is separately examined to confirm the trend of the derivative waveform slope. During a first loop the processor examines the window of the derivative waveform for a declining slope condition (SD) prior to the low point. The processor adds the values of the successively decreasing data values that exist prior to the low point sample index. A “true” SD trend condition exists if the values of at least 10 successive samples before the low point are successively lower.

If a true SD trend is not determined, the processor 50 examines the derivative waveform for an inclining slope trend (SU). The processor adds the values of the successively increasing data values that exist over the remainder of the window after the low point sample index. A “false” or SU trend condition exists if the values of at least 10 successive samples after the low point are successively higher.

Upon locating a low point sample index and qualifying the location in the window and slope trend of the waveform in the region of the sample, the processor 50 further analyzes the corresponding derivative waveform window to obtain a sum or score of several characteristics derived from the table containing the absolute value data for the samples of the derivative window. Specifically, the processor 50 develops a score value which is the sum of six values obtained from the following separately obtained parameters derived from the sampled data tables of the unfitted, fitted and derivative waveforms of FIGS. 13 and 14:

1. How many samples the curve was going down (SD)? The SD value is the value obtained during the above inspection of the derivative data immediately prior to the located low point but limited to a maximum value of 200.

2. How many samples the curve was going up (SU)? The SU value is the value obtained during the above “false” or inclining slope inspection of the derivative data immediately after the located low point but limited to a maximum value of 200.

3. Curve surface when going up after LP (LP)? The “curve surface” when going up after the calculated low point is determined by processing the data of the window of the fitted pressure data. Specifically the value of the low point is first subtracted from each of the window's sample data values, which causes the low point to be zero. The adjusted data points are next added and the sum is divided by the number of data points (i.e. window width). Lastly, the resulting quotient is multiplied by 200 to establish the importance of this parameter.

4. How steep the curve goes up after LP? How steeply the curve rises after the calculated low point is determined by processing the data of the window of the fitted pressure data after the calculated LP. Specifically, the trailing slope “m” is determined using the LabVIEW® linear fit coefficients function shown below with the low point as the start index and SU as the length and solving for “m”. The calculated slope m is multiplied by 1000 but limited to a maximum of 100.

F=mX+b

where F represents the output sequence best linear fit, X represents the input sequence, m is the slope, and b is the intercept.

5. How steep the curve goes down before LP? How steeply the curve declines before the low point is determined by processing the data of the window of the fitted pressure data after the calculated LP. Specifically, the leading slope “m” is determined using the foregoing LabVIEW® linear fit coefficients function with the start index as LP minus SD. The calculated slope “m” is negated to produce a positive value which is multiplied by 1000 and again limited to a maximum of 100.

6. What is the pressure drop? The pressure drop is calculated from the unfitted waveform data of FIG. 11 as the difference between the original unfitted maximum pressure of the sensing cuff 14 at the start deflation and the minimum pressure of the original unfitted pressure of the sensing cuff 14 for a window correlated to the fitted window. The difference is multiplied by 20 and limited to a maximum of 50. The result is allowed to be negative, if the pressure went up instead of down, and in which case the pressure drop value is add as a negative value to the score.

Lastly, the index or address of the calculated low point and the related score value is stored in a list or table and the next loop cycle is performed, until all loops are completed. When all the loops are completed, the resulting list is sorted and arranged by score value. The sample low point index containing the highest score is retained for further analysis as the point where the curve transitions from a down or declining slope to an up or inclining slope.

### Find Systolic Pressure

The foregoing processing steps essentially identify and locate the encircled region of interest at FIG. 11 and a preliminarily identified low point and corresponding sensed pressure at the sensing and occluding cuffs 14 and 16 of FIGS. 7 and 6. To confirm the authenticity of the preliminary LP index and with attention to FIGS. 12 and 13, the processor 50 next selects a subset of samples taken from the original DC sensor cuff pressure data samples of FIG. 13 around the selected and scored LP index. The start index of the subset is (“lowest point index” LP)−(sample rate×20)/deflation rate mmHg/sec. The window width is the [max of (sample rate×18)/deflation rate mmHg/sec and (length ramp up×0.9)]+(sample rate×20)/deflation rate mmHg/sec.

The processor 50 cleans up the selected subset by again applying the LabVIEW® median filter function to the previously filtered region of original data with a filter rank of 0.1 s. The re-filtered partial waveform is then evaluated and scored as before using a window width of 6s, a declining slope trend condition and a max curve fitting order of 7. The lowest point index of the partial waveform having the highest score is then retained.

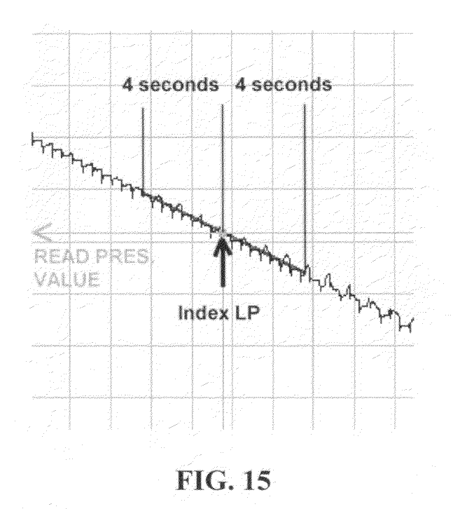

The processor 50 with attention to FIG. 15 lastly selects a subset of the DC occluding cuff pressures of FIG. 6 around the LP index determined above. The start index of this subset is (LP index−(sample rate×4)). The length of the sample subset is the (sample rate×8). Essentially, a subset of samples over eight seconds or 4 seconds before the LP index and 4 seconds after are analyzed to confirm the location of the final LP index.

The processor 50 particularly performs a linear fitting operation on the selected curve piece of FIG. 15 using the LabVIEW® general polynomial fitting function. The finally obtained LP index is correlated to the pressures measured for this partial curve and the pressure occurring at the final LP index is selected as the systolic pressure for the tested limb.

In a similar fashion, the processor processes the measured occluding and sensed pressures for each of the subject's limbs. Upon determining a systolic pressure for each limb, the processor 50 applies the general equation noted above for determining ABI.

$$A B I = \frac{Ankle Systolic Blood Pressure}{Brachial Systolic Blood {Pressure}^{*}}$$

$${}^{*} Highest systolic pressure found in left or right arm$$

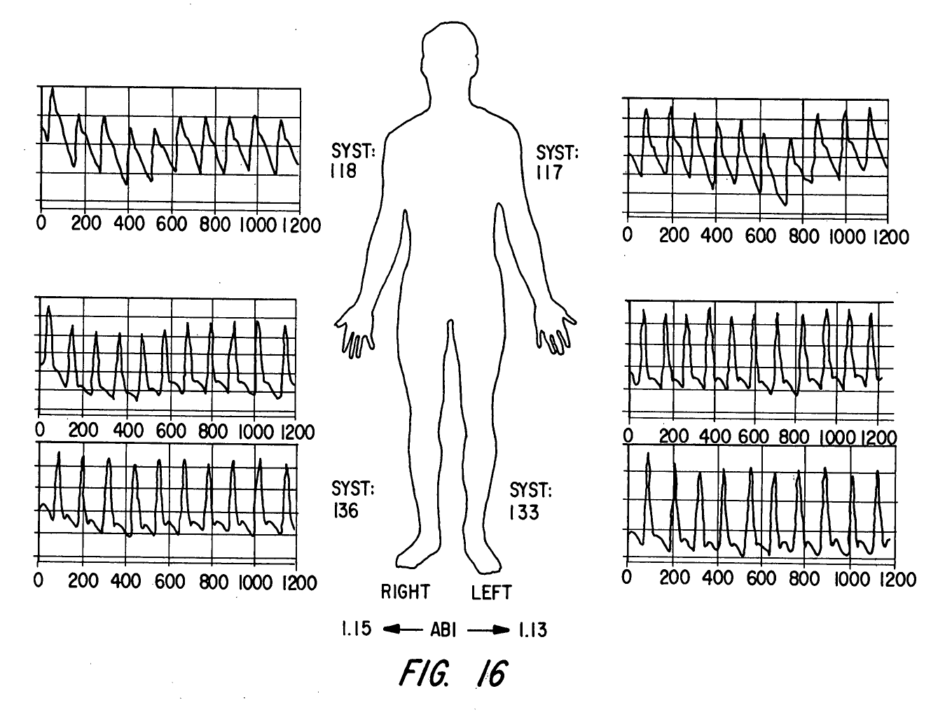

Right and left ABI values are calculated and are displayed at the monitor 52 and/or in a printed report, along with other pertinent test data for each limb such as the composite waveforms of FIGS. 8-10, reference FIG. 16. A technician can briefly evaluate the data for relevance and if necessary perform additional tests. The ultimately prepared report is submitted to an attending diagnostician as one of many inputs for further evaluation as to the subject's vascular condition and use in preparing a possible treatment plan.

While the invention has been described with respect to a preferred system assembly and alternative processing techniques, along with considered modifications and improvements thereto. It is to be appreciated still other system arrangements and processes may be suggested to those skilled in the art. The scope of the invention should therefore be construed broadly within the spirit and scope of the following claims.

What is claimed is:

1. Apparatus for determining a blood pressure of a subject comprising:
a) a pressure monitor adapted to be coupled to a limb of a test subject, wherein the monitor comprises 1) first and second inflatable cuffs and first and second pressure sensors respectively coupled to said first and second cuffs, 2) a compressor, 3) a deflation valve coupled to said first and second cuffs, 4) a first processor and wherein said first processor i) controls said compressor to inflate said first cuff to engage said first sensor to the limb and until blood flow through the limb is occluded and, ii) controls said compressor to inflate said second cuff to engage said second sensor to the limb at a location distal to said first cuff, iii) controls said deflation valve to deflate said first cuff in a plurality of steps, iv) stores data from said first sensor defining the pressure of said first cuff at occlusion and at each of said steps, and v) stores blood flow pressure data from said second sensor until termination of the deflation of the first cuff;
b) a wireless communication link;
c) a second processor coupled to said first processor via said communication link to receive data collected from said first and second sensors; and
d) wherein the second processor processes the data received from said first and second sensors by i) sampling the occluding pressure data from said first sensor and the blood flow pressure data from said second sensor in a filtering operation to obtain first and second tables of indexed sample data and whereby the sample data exhibits reduced artifact and noise variances, ii) sampling the blood flow pressure sample data from table two in a derivative function to obtain a third table of sampled derivative data including at least one value identifying at least one slope transition of a waveform defined by the sample data of table two from declining to inclining, iii) arithmetically scoring seriatim groupings of the filtered sample data of table two relative to a plurality of conditions of the waveform of the filtered sample values and relative to the derivative values of said table three to determine an arithmetic score defining a low point sample value where the at least one slope transition of the filtered blood flow pressure sample data changes from declining to inclining, iv) correlating the low point sample value of table two relative to the unfiltered pressure data measured by the sensor one to determine a systolic pressure when blood flow returns to the monitored limb.

2. Apparatus as set forth in claim 1 wherein said first and second sensors are integrated into said first and second cuffs and wherein the occluded first cuff is deflated in a linear fashion with a constant pressure drop at each deflation step.

3. Apparatus as set forth in claim 1 wherein during inflation of said first and second cuffs the compressor is gradually slowed as an occlusion pressure for said first cuff and a sensor retention pressure for said second cuff is neared.

4. Apparatus as set forth in claim 1 wherein the sampled derivative data of table three includes a plurality of sample values identifying a plurality of slope transitions from declining to inclining and inclining to declining and which sample values can coincide with motion artifacts, signal or vascular noise.

5. Apparatus as set forth in claim 1 wherein the pressure monitor identifies the limb of the subject to which it is attached and re-learns the limb with each new test.

6. Apparatus as set forth in claim 1 including a DC voltage charging device for recharging a contained power supply of a plurality of said pressure monitors between tests.

7. A method for determining a systolic blood pressure of a subject comprising:
a) coupling a pressure monitoring device to a limb of a test subject, wherein the monitoring device comprises 1) a first inflatable cuff having a first pressure sensor coupled to the limb and a second inflatable cuff having a second pressure sensor coupled to the limb distal to said first sensor, 2) a compressor, 3) a deflation valve coupled to said inflatable cuff, 4) a first processor and wherein said first processor i) controls said compressor to inflate said first cuff to engage said first sensor to the limb and until blood flow through the associated limb is occluded and to inflate said second cuff to engage said second sensor to the limb, ii) controls said deflation valve to deflate said first cuff in a plurality of deflation steps until blood flow returns to the limb, iii) stores pressure data from said first sensor at occlusion and at each deflation step until a test is terminated, and iv) stores blood flow pressure data from said second sensor until termination of the deflation of said first cuff;
b) enabling a wireless communication link;
c) coupling a second processor to said first processor via said communication link to receive the data collected from said first and second sensors; and
d) processing the received first and second sensor data at said second processor by i) sampling the occluding pressure data from said first sensor and the blood flow pressure data from said second sensor in a filtering operation to obtain first and second tables of indexed sample data and whereby the sample data exhibits reduced artifact and noise variances, ii) sampling the blood flow pressure sample data from table two in a derivative function to obtain a third table of sampled derivative data including at least one value identifying at least one slope transition of a waveform defined by the sample data of table two from declining to inclining, iii) arithmetically scoring seriatim groupings of the filtered sample data of table two relative to a plurality of conditions of the waveform of the filtered sample values and relative to the derivative values of said table three to determine an arithmetic score defining a low point sample value where the at least one slope transition of the filtered blood flow pressure sample data changes from declining to inclining, iv) correlating the low point sample value of table two relative to the unfiltered pressure data measured by the sensor one to determine a systolic pressure when blood flow returns to the monitored limb.

8. A method as set forth in claim 7 wherein a plurality of monitoring devices are mounted to a plurality of limbs, wherein the second processor determines a systolic pressure for each limb and wherein the second processor determines a ratio of the systolic pressure for a limb near a subject's heart to a systolic pressure determined for another limb more remote from the subject's heart to define an index value representative of the occlusion of the blood vessels contained in the remote limb.

9. A method as set forth in claim 7 wherein the arithmetic score value is computed from a plurality of values obtained from a class of parameters derived from the pressure data measured by the sensor two and the sample data of tables one, two and three and which class of parameters can include:
i) a value correlated to declining slope data of table three immediately prior to the determined low point,
ii) a value correlated to inclining slope data of table three immediately after the determined low point,
iii) a value correlated to inclining slope data of table two immediately after the determined low point and adjusted for the sample value of the low point,
iv) a value correlated to inclining slope data of table two immediately after the determined low point and indicative of the rate of change of the inclining slope,
v) a value correlated to declining slope data of table two immediately before the located low point defining the rate of change of the inclining slope, and
vi) a value correlated to a pressure drop or difference between the maximum and minimum values of the unfiltered pressure data of sensor two.

10. A method as set forth in claim 9 wherein the highest score value determines the low point sample value and wherein the second processor upon determining the low point sample value selects a narrowed range of sample values of table two containing the low point sample value, performs a second filtering of the narrowed range and a second arithmetic scoring of the narrowed range to determine a refined low point sample value and the index to which is correlated to the unfiltered data of sensor one to determine the systolic pressure.

11. A method as set forth in claim 10 wherein the second processor during the correlation of the refined low point sample value of table two to the filtered sample data of table one performs a linear fitting operation on a narrowed range of the unfiltered pressure data from sensor one that includes the pressure at the refined low point sample value to determine a final low point index and the corresponding sensor one pressure which is selected as the systolic pressure.

12. A method for determining a blood pressure index of a subject comprising:
a) coupling a plurality of pressure monitoring devices to the arms and at least one leg of a test subject, wherein each monitoring device comprises 1) first and second inflatable cuffs and first and second pressure sensors respectively coupled to said first and second cuffs, 2) a compressor, 3) a deflation valve coupled to said first and second cuffs, 4) a first processor and wherein said first processor of each pressure monitoring device independently i) controls said compressor to inflate said first cuff to engage the first sensor to the associated limb and until blood flow through the associated limb is occluded, ii) controls said compressor to inflate said second cuff to engage said second sensor to the associated limb at a location distal to said first cuff, iii) controls said deflation valve to deflate said first cuff in a plurality of deflation steps until flow returns to the limb, iv) stores data from said first sensor defining the pressure of said first cuff at occlusion and at each deflation step, and v) stores data from said second sensor defining the blood flow through the limb until termination of the stepped deflation of said occluding cuff;
b) enabling a wireless communication link;
c) coupling a second processor to said first processor of each monitoring device via said communication link to receive the stored data of said first and second sensors of each monitoring device; and
d) processing the received first and second sensor data from each monitoring device at said second processor by respectively i) sampling the occluding pressure data from said first sensor and the blood flow pressure data from said second sensor in a median filtering operation to obtain first and second tables of indexed sample values and whereby the pressure sample data exhibits reduced artifact and noise variances, ii) sampling the blood flow pressure sample data of table two in a derivative function to obtain a third table of derivative values including at least one value identifying at least one slope transition of a waveform defined by the sample data of table two from declining to inclining, iii) arithmetically scoring seriatim groupings of said median sample data of table two relative to a plurality of conditions of the waveform defined by the table two sample values and relative to the derivative values of said table three to determine an arithmetic score defining a low point sample value where the at least one slope transition of the median blood flow pressure sample data of table two changes from declining to inclining, iv) correlating the low point sample value of table two relative to the occluding cuff pressure data of table one and the related pressure measured by the sensor two to determine a systolic pressure for the monitored limb, v) computing a ratio of the of the systolic pressures determined by the second processor from the plurality of monitoring devices wherein the highest systolic pressure determined for the arms is contrasted to the systolic pressure determined for the at least one leg to define an index value representative of the occlusion of the blood vessels contained in the leg.

13. A method as set forth in claim 12 wherein the arithmetic score value is computed from a plurality of values obtained from a class of parameters derived from the pressure data measured by the sensor two and the sample data of tables one, two and three and which class of parameters can include:
i) a value correlated to declining slope data of table three immediately prior to the determined low point,
ii) a value correlated to inclining slope data of table three immediately after the determined low point,
iii) a value correlated to inclining slope data of table two immediately after the determined low point and adjusted for the sample value of the low point,
iv) a value correlated to inclining slope data of table two immediately after the determined low point and indicative of the rate of change of the inclining slope,
v) a value correlated to declining slope data of table two immediately before the located low point defining the rate of change of the inclining slope, and
vi) a value correlated to a pressure drop or difference between the maximum and minimum values of the unfiltered pressure data of sensor two.

14. A method as set forth in claim 12 wherein each pressure monitoring device re-identifies the limb to which it is attached to said second processor with each successive test.
